(19) United States
(12) Patent Application Publication
(10) Pub. No.: US 2025/0101530 A1
(43) Pub. Date: Mar. 27, 2025

(54) SERIAL SAMPLING DIAGNOSTICS

(71) Applicant: Mercy Bioanalytics, Inc., Waltham, MA (US)

(72) Inventor: Stanley Lapidus, Denver, CO (US)

(21) Appl. No.: 18/894,448

(22) Filed: Sep. 24, 2024

Related U.S. Application Data
(60) Provisional application No. 63/585,038, filed on Sep. 25, 2023.

(51) Int. Cl. C12Q 1/6886; C12Q 1/6841

(52) U.S. Cl. CPC C12Q 1/6886; C12Q 1/6841; C12Q 2600/156

(57) ABSTRACT

The invention provides methods of diagnostic screening for early disease detection by analyzing samples from a subject at multiple points in time

### FIELD OF THE INVENTION

The invention relates to diagnostic screening and methods of detecting disease biomarkers.

### BACKGROUND

Diagnostic screenings are a routine part of basic healthcare. They are used to detect anything from cancer to pregnancy and even physical injury. These diagnostic screenings can comprise anything from biopsies of tissue and bodily fluid to MRIs, and more.

Typically, diagnostic screenings are performed annually, semiannually, or for management of a known condition. This allows healthcare practitioners to track the progression of certain risk factors for disease and accurately diagnose and treat disease. However, especially in early stages, some biomarkers may be present stochastically. Thus, a single point sample may fail to detect certain biomarkers, resulting in false-negative screening or diagnosis.

Diagnostic and screening assays are often based on biomarker detection. Most commonly, diagnostic tests are samples obtained from tissue or body fluid. These tests are commonly performed as part of an annual physical examination or for monitoring of a known condition (e.g., blood glucose monitoring for diabetes). Diagnostic screening can be useful for assessing changes in many key health factors, such as cholesterol levels, liver function, etc. This approach offers a chance to monitor basic health indicators.

A problem with conventional screening is that some disease biomarkers appear only sporadically or stochastically, especially in the earliest stages of a disease. Conventional testing for a biomarker that is produced or present stochastically may fail to detect the biomarker when a single point of detection is used.

In addition, many disease biomarkers are detectable through extracellular vesicles. Extracellular vesicles are lipid bilayer-delimited particles that are naturally released from almost all types of cells. Extracellular vesicles can carry a cargo of proteins, nucleic acids, lipids, metabolites, and even organelles from their parent cell. The extracellular vesicles play a key role in intercellular signaling, immune response, and cellular waste management. They often contain disease biomarkers. These can be tested, and biomarkers can be detected at a much earlier time than normal. Additionally, extracellular vesicles are also often found to be circulating in abundance during early-stage cancer.

Early-stage detection and identification of a disease is paramount to an effective treatment and recovery. Thus, there is a need for an improved method of diagnostic screening that allows for earlier detection of disease in a patient.

### SUMMARY

The present invention provides improved methods for diagnostic screening. In particular, the invention provides methods for the detection of a biomarker that appear stochastically in a tissue or body fluid sample. The present invention encompasses any method in which a sample is obtained from a clinical subject at two or more time points. Assays are performed to identify one or more biomarker(s) in the samples. Thus, if a particular biomarker or biomarkers are produced only sporadically, the assay will identify them through stochastic sampling methods of the invention. A positive screen is defined as the presence of the assayed biomarker in at least one of the serial samples. Thus, the invention increases the likelihood of detecting biomarkers that appear only stochastically and may be missed in conventional screening and testing methods.

In certain embodiments, the invention includes methods for diagnostic screening comprising obtaining a tissue or body fluid sample at two or more time points, performing an assay to identify a biomarker in at least one of the samples, wherein the biomarker is produced stochastically in the sample, and identifying a positive screen as the presence of the biomarker in at least one of the samples.

According to an embodiment of the present invention, the biomarker is produced sporadically in vivo during a first phase of a disease and is produced consistently in vivo during a subsequent phase of the disease. Alternatively, the biomarker is produced early in disease progression but is under the detection limit until some later time. The biomarker may also be one that is produced or expressed in the sample inconsistently during the earliest stages of the disease.

According to another embodiment of the present invention the sampling timepoints may be separated by from about three hours to about six months. The number and spacing of sampling will depend on the disease being screened and the biomarkers being detected. In general, however, assays are performed a sufficient number of times to overcome stochastic in vivo production of the biomarker, thus reducing or eliminating false negative results.

Biomarkers for use in the invention include any biomarker that is produced in vivo and is indicative of, or in response to, a disease. The disease may be an infectious disease an environmental disease, a disease caused by exposure to a toxin or immunogen, or a disease of genomic origin, such as cancer, multiple sclerosis, and the like. In specific embodiments of the invention, the biomarker is indicative of precancer, for example, a colonic polyp.

Biomarkers for use in the invention include nucleic acids (including mutations, such as single nucleotide polymorphisms, deletions, rearrangements, loss of heterozygosity, translocations, copy number variation, structural variants and the like), proteins (including enzymes, antibodies, posttranslational modifications (e.g., phosphorylation, ubiquitinylation, glycosylation and others), carbohydrates, membrane-bound receptors and their ligands, hormones (including steroid hormones and peptides), and other markers associated with, or suspected to be associated with, a condition.

Samples for use in the invention include any tissue or body fluid sample. In preferred embodiments, the sample is selected from blood, urine, stool, cerebrospinal fluid (CSF), tumor biopsy, saliva, biopsy material, and lymphatic fluid.

### DETAILED DESCRIPTION

The present invention discloses methods for diagnostic screening of biomarkers indicative of disease. The invention accounts for the fact that biomarkers may be produced stochastically in tissue and/or body fluid. By sampling at multiple time points, methods of the invention detect the presence and/or activity of biomarkers that may not be detected in single-point assays due to variations in their expression and/or activity over time.

Embodiments of the present invention utilize serial sampling to detect disease biomarkers. In order to do so, samples of a body tissue or fluid are taken at multiple different time points. Samples can be taken at intervals separated by minutes, hours, days, weeks, months, or any combination thereof sufficient to maximize the chance of detection of the biomarker.

Once the sample has been gathered it must be analyzed in order to detect the target biomarker. Embodiments of the present invention may also use various methods for performing an assay and detecting a positive or negative screen for the presence of a biomarker in at least one of the gathered samples.

Biomarkers include, but are not limited to, DNA, RNA, protein, lipids, carbohydrates, single-nucleotide polymorphisms (SNPs), protein specific antigens (PSA), specific cell types, antigens/antibodies, and more.

In some embodiments, an assay includes two steps for the detection and identification of a biomarker in or on an extracellular vesicle. The first step comprises a capture assay in which one or more biomarker signature is (are) used to target extracellular vesicles that may contain the target biomarker. Here, capture reagents, microfluidic devices, and other diagnostic tools may be used to capture one or more extracellular vesicles. The second step comprises a detection assay. This step may comprise the utilization of various laboratory tests to detect the presence of a biomarker in the captured extracellular vesicles. Depending on the biomarker in question, various detection assays may be used.

In some embodiments, methods of detecting one or more biomarkers include detecting a target biomarker signature comprising proteins, glycans, or proteoglycans (including, e.g., but not limited to a protein with a carbohydrate or glycan moiety). Exemplary protein-based methods include, but are not limited to, proximity ligation assay, mass spectrometry (MS) and immunoassays, such as immunoprecipitation; Western blot; ELISA; immunohistochemistry; immunocytochemistry; flow cytometry; and immuno-PCR. In some embodiments, an immunoassay is a chemiluminescent immunoassay. In some embodiments, an immunoassay is a high-throughput and/or automated immunoassay platform.

In some embodiments, detection methods comprise contacting a sample with one or more antibody or antibody fragment directed against a biomarker of interest. In some embodiments, such methods also comprise the use of detectable labels.

In some embodiments, methods comprise detecting nucleic acids and nucleic acid variants. Exemplary nucleic acid-based methods of detecting one or more provided markers include, but are not limited to, performing nucleic acid amplification methods, such as polymerase chain reaction (PCR), reverse transcription polymerase chain reaction (RT-PCR), transcription-mediated amplification (TMA), ligase chain reaction (LCR), isothermal amplification methods (e.g., loop-mediated isothermal amplification), strand displacement amplification (SDA), and nucleic acid sequence-based amplification (NASBA), and next generation sequencing technologies. In some embodiments, a nucleic acid-based methods include detecting hybridization between one or more nucleic acid probes and one or more nucleotide sequences that encode a biomarker of interest. In some embodiments, the nucleic acid probes are each complementary to at least a portion of one of the one or more nucleotide sequences. In some embodiments, the nucleotide sequences include DNA (e.g., cDNA). In some embodiments, the nucleotides of interest include RNA (e.g., mRNA, snRNA, miRNA, siRNA, orphan noncoding RNA, long noncoding RNA, or piwi-interacting RNA). In another aspect, the invention includes direct detection of peptides and proteins, including those translated endogenously.

Embodiments of the present invention may also be used for the detection of cell-free fetal DNA (cffDNA). cffDNA consists of placental microparticles which shed into the maternal blood circulation. cffDNA exists at low concentrations (around 12%) in maternal blood and is often used to provide early diagnosis of fetal conditions.

According to another embodiment of the invention, the biomarker is indicative of a precancerous lesion. This precancerous lesion may further be associated with an epithelial lining such as, a colonic polyp. In its earliest stages, it is unlikely that evidence of this would be found on a singular diagnostic test. However, using the present invention, a blood sample, for example, could be taken at two or more time points. The sample would be assayed to find ctDNA and/or biomarkers contained in extracellular vesicles from site of the colonic polyp. If ctDNA and/or an extracellular vesicle containing a biomarker were present in any of the samples taken, it would indicate a positive cancer test. This would then allow doctors to begin treatment at the earliest stage possible.

Embodiments of the invention further provide a method for diagnostic screening wherein the biomarker is produced sporadically in situ during the first phase of a disease and then is produced consistently in situ during a second subsequent phase of the disease. The biomarker may further be expressed inconsistently during the first phase of the disease.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for diagnostic screening, the method comprising the steps of:
obtaining a tissue or body fluid sample from a subject at two or more time points;
performing an assay to identify a biomarker in at least one of the samples, wherein the biomarker is detectably present stochastically in the sample; and
identifying a positive screen as the presence of the biomarker in at least one of the samples.

2. A diagnostic screening assay comprising the steps of:
performing an assay to detect a biomarker in a tissue or body fluid sample at a first time;
performing the assay on subsequent tissue or body fluid samples at a number of additional times sufficient to ensure detection of a biomarker that is produced stochastically in said tissue or body fluid.

3. A method for obtaining a representative tissue or body fluid sample, the method comprising the steps of:
obtaining a plurality of tissue or body fluid samples at a number of different times sufficient to detect a biomarker that is produced in the samples sporadically.

4. The method of claim 1, wherein the biomarker is produced sporadically in vivo during a first phase of a disease and is produced consistently in situ during a subsequent phase of a disease.

5. The method of claim 1, wherein the disease is cancer or precancer.

6. The method of claim 1, wherein the sample is selected from blood, stool, CSF, tumor biopsy, saliva, and lymphatic fluid.

7. The method of claim 1, wherein the biomarker is indicative of a precancerous lesion.

8. The method of claim 7, wherein the precancerous lesion is associated with an epithelial lining.

9. The method of claim 8, wherein the lesion is a colonic polyp.

10. The method of claim 1, wherein the time points are separated by from about one day to about three days.

11. The method of claim 1, wherein the assay is performed a sufficient number of times to account for false negative results.

12. The method of claim 1, wherein the biomarker is produced or expressed in the sample inconsistently during early stages of a disease.
